(19) United States
(12) Patent Application Publication
(10) Pub. No.: US 2021/0187495 A1
(43) Pub. Date: Jun. 24, 2021

(54) Multi-compartment biological fluid storage device

(71) Applicant: Texas Heart Institute, Houston, TX (US)

(72) Inventors: Doris Anita Taylor, Houston, TX (US); Luiz C. Sampaio, Pearland, TX (US); Lourdes Isabel Chacon, Houston, TX (US)

(21) Appl. No.: 16/625,560

(22) PCT Filed: Jun. 22, 2018
(86) PCT No.: PCT/US18/39047

§ 371 Date: Dec. 20, 2019

Related U.S. Application Data
(60) Provisional application No. 62/523,631, filed on Jun. 22, 2017.

(51) Int. Cl. B01L 3/00; C12M 1/00

(52) U.S. Cl. CPC B01L 3/505; B01L 2300/0681; B01L 2200/026; C12M 45/22

(57) ABSTRACT

An storage container for a biological fluid includes, for example, a sequence of elongated compartments interconnected by passageways. The sequence forms a single continuous tube or ribbon having a first end, to which a first port is optionally connected, and a second end, to which a second port is also optionally connected. After filling the container with a biological fluid, multiples closures are formed across the single continuous tube or ribbon, for example across each passageway, to seal the compartments.

### BACKGROUND

This disclosure relates generally to methods and apparatuses for storing a biological fluid in a device that may be suitable for cryopreservation of the biological fluid. More particularly, this disclosure relates to a multi-compartment bag for biological fluid storage that may facilitate the filling and the sealing of the compartments and to methods of using such a multi-compartment bag.

Biological fluids, such as the ones comprising cells, genes, proteins, are currently stored in aliquots in preparation for cryopreservation. Decreasing the volume of the aliquots usually increases the survival rate of the cells. Furthermore, decreasing the cross-sectional area of the aliquots usually ensures a uniform and homogeneous cell density through the aliquots. Therefore, these biological fluids are preferably stored in small aliquots, wherein the aliquots have a volume and/or cross-section area that is sufficiently small for being suitable for cryopreservation.

In general, a single aliquot, which may have a volume of approximately two cubic centimeters (or two milliliters), is too small to store all of the cells that may be extracted from a typical blood sample or other sample. Therefore, a typical sample is stored in a device having multiple compartments that may then be sealed from the other compartments of the device. Each compartment of the device stores an aliquot of the sample.

In some known devices, each compartment may consist of a closed chamber that stems from a common pathway connected to a fill line tubing. After filling, the compartments may be sealed from each other by fusing the opposite walls of the common pathway together, typically by applying heat. These known devices may be difficult to fill or to seal.

Thus, there is a continuing need in the art for methods and apparatus for biological fluid storage that can facilitate the filling and the sealing of multiple compartments.

### BRIEF SUMMARY OF THE DISCLOSURE

The disclosure describes a storage bag for a biological fluid.

In some embodiments, the storage bag may comprise a bottom sheet and a top sheet superimposed on the bottom sheet. The top sheet and/or the bottom sheet may be flexible. A first portion of the top sheet may be sealed against a second, corresponding portion of the bottom sheet. The first portion and second portion may enclose a ribbon having a first end and a second end. The ribbon may be continuous between the first end and the second end. For example, the ribbon may form a single tube, and/or may form a sequence of elongated compartments that may be interconnected by passageways. In some embodiments, the storage bag may comprise a sequence of elongated compartments which may be interconnected by passageways. The sequence may form a single continuous tube having a first end and a second end. Each passageway may be connected to an extremity of a first elongated compartment and to an extremity of a second elongated compartment. Preferably, each passageway may only be connected to the extremity of the first elongated compartment and to the extremity of the second elongated compartment.

The storage bag may comprise a first port, which may be connected across the first end of the ribbon and/or the first end of the continuous tube. A valve may be connected to the first port. The valve may comprise a Luer lock.

The storage bag may comprise a second port, which may be connected across the second end of the ribbon and/or the second end of the continuous tube. A filter may be connected to the second port. A valve may also be connected to the second port. The valve may comprise a Luer lock.

The storage bag may comprise a first means for closure between the top sheet and the bottom sheet and/or a first means for closure across one of the passageways. In some embodiments, the first means may comprise a first male rib, which may be attached to one of the top sheet and the bottom sheet along a first cross-section of the ribbon, or which may be attached to a wall portion of one of the passageways along a first cross-section of the continuous tube. The first means may also comprise a first female channel, which may be attached to the other of the top sheet and the bottom sheet along the first cross-section of the ribbon, or which may be attached to a complementary wall portion of the one of the passageways along the first cross-section of the continuous tube. Upon mating of the first female channel with the first male rib, the first male rib and the first female channel may form a first closure between the top sheet and the bottom sheet, or across the one of the passageways. The first closure may be fluid-tight.

The storage bag may comprise a second means for closure between the top sheet and the bottom sheet and/or a second means for closure across the one of the passageways. In some embodiments, the second means may comprise a second male rib, which may be attached to one of the top sheet and the bottom sheet along a second cross-section of the ribbon, or which may be attached to a wall portion of the one of the passageways along a second cross-section of the continuous tube. The second means may also comprise a second female channel, which may be attached to the other of the top sheet and the bottom sheet along the second cross-section of the ribbon, or which may be attached to a complementary wall portion of the one of the passageways along the second cross-section of the continuous tube. Upon mating of the second female channel with the second male rib, the second male rib and the second female channel may form a second closure between the top sheet and the bottom sheet, or across the continuous tube.

The first cross-section and the second cross-section may preferably be offset from each other. In some embodiments, the first cross-section may be adjacent to the second cross-section.

The disclosure also describes a method of storing a biological fluid.

The method may comprise the step of providing a storage bag. In some embodiments, the storage bag may include a bottom sheet and a top sheet superimposed on the bottom sheet. A first portion of the top sheet may be sealed against a second, corresponding portion of the bottom sheet. The first portion and second portion may enclose a ribbon having a first end and a second end. The ribbon may be continuous between the first end and the second end. In some embodiments, the storage bag may comprise a sequence of elongated compartments which may be interconnected by passageways. The sequence may form a single continuous tube having a first end and a second end.

The method may further comprise the step of flowing the biological fluid into a first port connected across the first end of the ribbon.

The method may further comprise the step of venting air through a second port connected across the second end of the ribbon.

The method may further comprise the step of forming a first closure. The first closure may be formed by mating a first male rib with a first female channel. The first male rib may be attached to one of the top sheet and the bottom sheet along a first cross-section of the ribbon, or may be attached to a wall portion of one of the passageways along a first cross-section of the continuous tube. The first female channel may be attached to the other of the top sheet and the bottom sheet along the first cross-section of the ribbon, or may be attached to a complementary wall portion of the one of the passageways along the first cross-section of the continuous tube. Alternatively, the first closure may be formed by fusing the top sheet and the bottom sheet along a first cross-section of the ribbon and/or the continuous tube.

The method may further comprise the step of forming a second closure by mating a second male rib with a second female channel. The second male rib may be attached to one of the top sheet and the bottom sheet along a second cross-section of the ribbon or may be attached to a wall portion of the one of the passageways along a second cross-section of the continuous tube. The second female channel may be attached to the other of the top sheet and the bottom sheet along the second cross-section of the ribbon, or may be attached to a complementary wall portion of the one of the passageways along the second cross-section of the continuous tube. Alternatively, the second closure may be formed by fusing the top sheet and the bottom sheet along a second cross-section of the ribbon and/or the continuous tube.

The method may further comprise the step of severing the storage bag between the first cross-section and the second cross-section. Accordingly, the first cross-section and the second cross-section may preferably be offset from each other. In some embodiments, the first cross-section may be adjacent to the second cross-section.

The method may further comprise the step of freezing the biological fluid.

### BRIEF DESCRIPTION OF THE DRAWINGS

For a more detailed description of the embodiments of the disclosure, reference will now be made to the accompanying drawings, wherein:

FIG. 1 is a schematic of a storage container or bag according to a first embodiment, wherein the compartments are side by side, and wherein passageways interconnecting the compartments are disposed on alternate sides of the container;

FIG. 1A is a schematic of a passageway interconnecting two compartments of the storage container or bag shown in FIG. 1, wherein the passageway includes at least a pair of male ribs and a pair of female channels for forming at least two closures, one on each side of a separation mark;

FIG. 2 is a schematic of the storage container or bag shown in FIG. 1 after separation of the compartments;

FIG. 3 is a schematic of a storage container or bag according to a second embodiment, wherein the compartments are U-shaped;

FIG. 4 is a schematic of a storage container or bag according to a third embodiment, wherein the compartments are aligned; and

FIG. 5 is a schematic of a storage container or bag according to a fourth embodiment, wherein the compartments are alternatively aligned or side by side.

### DETAILED DESCRIPTION

It is to be understood that the following disclosure describes several exemplary embodiments for implementing different features, structures, or functions of the invention. Exemplary embodiments of components, arrangements, and configurations are described below to simplify the disclosure; however, these exemplary embodiments are provided merely as examples and are not intended to limit the scope of the invention. Additionally, the disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various Figures. Finally, the exemplary embodiments presented below may be combined in any combination of ways, i.e., any element from one exemplary embodiment may be used in any other exemplary embodiment, without departing from the scope of the disclosure.

All numerical values in this disclosure may be approximate values unless otherwise specifically stated. Accordingly, various embodiments of the disclosure may deviate from the numbers, values, and ranges disclosed herein without departing from the intended scope. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

As one skilled in the art will appreciate, various entities may refer to the same component by different names, and as such, the naming convention for the elements described herein is not intended to limit the scope of the invention, unless otherwise specifically defined herein. Further, the naming convention used herein is not intended to distinguish between components that differ in name but not function.

The disclosure provides several embodiments of devices for storing a biological fluid. The devices may be used to cryopreserve multiple fractions or aliquots of the biological fluid. For example, the disclosure describes a container or bag that improves the process of storing, for example, a fluid suspension of cells extracted from blood, bone marrow, or fat, or adipose tissues. Alternatively of additionally, the container or bag can be used with other biological fluid containing a cell or non-cell material (e.g., containing plasma/serum, exosomes, vesicles, genes, RNA molecules, DNA molecules, proteins, viruses, etc. . .) In particular, the container or bag may be used for cryopreservation of biological fluid samples.

In some embodiments, the container or bag comprises an inlet port for injecting or infusing a biological fluid into the container. The inlet port may be provided with a Luer lock or an equivalent valve. The container or bag also comprises an outlet port. The outlet port may also be provided with a Luer lock or an equivalent valve for allowing air to be removed from the container as the biological fluid sample is injected or infused into the container. The container or bag preferably comprises several small compartments interconnected by passageways.

The biological fluid may be injected or infused via a single inlet port into a flexible, multi-compartment storage container or bag. The biological fluid flows sequentially into the small compartments. After all the sample of biological fluid has been inserted or infused in the bag, it may occupy all or less than all of the compartments.

Once the container or bag is filled via the single port, it may provide efficient separation of any biological fluid sample into multiple aliquots, which may then be individually processed, frozen, stored, thawed, or used without compromising or contaminating the other aliquots of the sample. Thus, the compartments that are occupied may then be sealed off after filling. Each small compartment can be separated from the others for individual processing, or use.

Each compartment may include an individual Luer lock communicating with an interior volume of the compartment. The purpose of the Luer locks may be to provide individual access to the interior volume of the compartments after the compartments are sealed off and separated from one another. The Luer lock may be the preferred valve fitting, however equivalent valve fittings may alternatively be used.

The storage container or bag further includes passageways, initially used for fluid communication between adjacent compartments during filling of the storage container. After filling, the passageways between the compartments may be sealed, creating, for example, five noncontiguous, fluid-filled compartments that can be separated for individual thawing, processing, storage, or use. In some embodiments, the passageways for fluid communication between the compartments may be located on alternate sides of the compartments to provide a flow of the biological fluid along a continuous, folded ribbon consisting, in some embodiments, of the sequence of compartments interconnected by the passageways.

In some embodiments, a storage bag may be made from a bottom sheet and a top sheet superimposed on the bottom sheet, the bottom sheet and the top sheet being made of flexible material. A first portion of the top sheet is sealed against a second, corresponding portion of the bottom sheet. As such, the first portion of the top sheet and the second portion of the bottom sheet may enclose a ribbon, which includes unsealed portions of the top sheet and the bottom sheet. As used herein, a ribbon means an essentially flat, narrow and long tubular, which may be straight or folded. The ribbon is continuous between its end or extremities.

Design variations of the storage container or bag can include but are not limited to, varying sizes of the individual compartments: each compartment may have a volume of one cubic centimeter, two cubic centimeters, five cubic centimeters, or ten cubic centimeters. The sizes of the individual compartments may preferably but not necessarily be essentially identical. Other design variations can include the way used to seal off the passageways between the compartments after filling. Sealing may be performed by fusing the material of opposite walls of the storage container by applying heat or pressing a male rib attached to one wall of the storage container into a female channel attached to an opposite wall of the storage container, or a combination of fusing and pressing. Still, other design variations can include replacing the valve provided at the outlet port by a sealed compartment under negative pressure (i.e., under partial vacuum) that may be used to drive the biologic fluid into the storage container or bag.

Optionally, the storage container or bag may include, connected to the outlet port, one or more filters having sizes between 0.08 micron and 0.22 micron. The one or more filters may be used alone or in stacks of filters having decreasing sizes to provide a barrier against any bacteria while compartments of the storage container are being filled sequentially and air in the compartments is being vented.

Unlike other known multi-compartment storage bags for cord blood storage that require each compartment to be filled separately before being stored as a unit, the devices for storing a biological fluid described herein can accommodate a single injection of homogenous biological fluid which flows sequentially into several separate, discrete compartments. Each compartment can be separated for individualized processing, storage, and use. The single-filling device may advantageously decrease the possibility of cross-contamination of the biological fluid by decreasing its contact with the external environment. Less than all of the compartments provided by the device may be filled before the compartments are sealed off

The disclosure also describes a method to store samples of a biological fluid, for example, a fluid containing cellular elements extracted from blood. The method permits efficiently freezing the samples in small aliquots in a way that promotes sample homogeneity. The cross-sectional area of each aliquot is small enough (e.g., between half a square centimeter and one and a half square centimeter) to allow uniform cell presence through all regions of the sample. Furthermore, the volume of each aliquot is sufficiently small (e.g., smaller than approximately five cubic centimeters) to increase cell survival percentage after freezing and thawing.

In some applications, the devices described herein may be used so that each separate compartment will contain a similar volume of mononuclear cells or buffy coat suspended in cryopreservation medium. Each individual compartment can be separated and used, processed, stored, or cryopreserved separately. Each individual compartment may be fitted with a Luer lock or equivalent valve fitting to facilitate access to its contents after separation.

For example, in one example application, a one hundred and fifty cubic centimeter sample of whole blood is centrifuged before or after removal of red blood cells. The cellular layer, containing an estimated fifty million mononuclear cells, may be suspended in ten cubic centimeters of cryopreservation liquid medium. The ten cubic centimeter solution may be injected into the inlet port and dispersed in sequence into five interconnected compartments of approximately two cubic centimeters each. A port on the distal end of the device may comprise a Luer lock or other valve fitted with a filter to prevent contamination, and to allow the release of air as the compartments fill with the solution. In another embodiment, the compartments may contain up to twenty-five cubic centimeters of a biological fluid in total after filling, separable in aliquots or fractions of approximately five cubic centimeters.

In some applications, the devices for storing a biological fluid described herein may be used to separate a cellular suspension extracted from peripheral blood, bone marrow, fat, or adipose tissue into smaller, homogeneous aliquots to avoid thawing the whole sample when only a few portions of the biologic fluid sample is needed. The device may advantageously reduce handling, optimize cryopreservation, and/or increase cell preservation and viability. One aliquot can then be used while keeping the other aliquots in a safe, cryopreserved stage for potential future use.

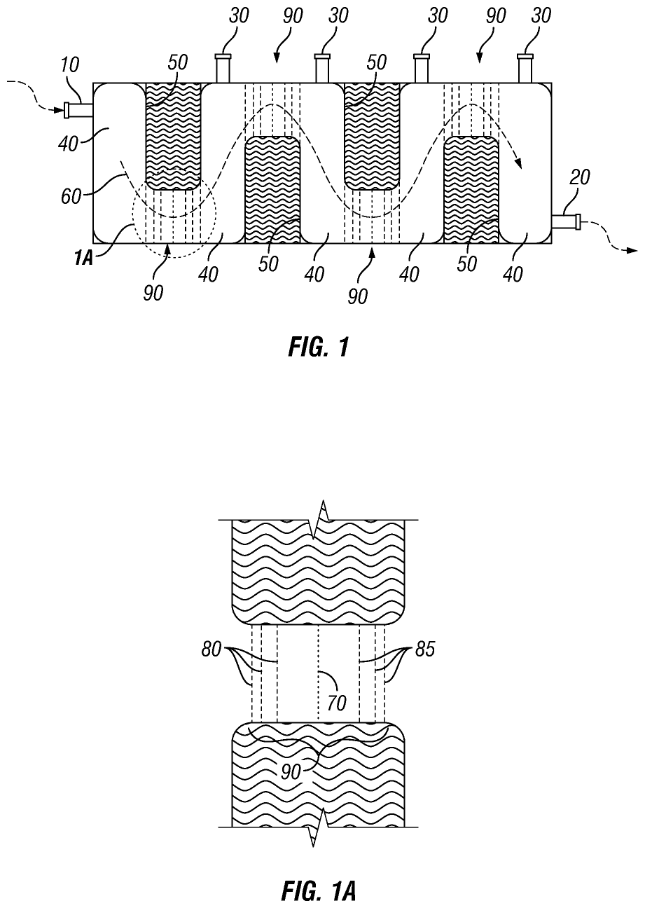

Referring initially to FIG. 1, a storage container or bag comprises five compartments 40, interconnected by four passageways 90. Together, the five compartments 40 and the four passageways 90 form a folded ribbon which is continuous between an inlet port 10 and an outlet port 20. The storage container or bag may be made by sealing a first portion of a top flexible sheet against a second, corresponding portion of a bottom flexible sheet along a boundaries 50 which encloses the continuous ribbon. Accordingly, in use, biological fluid flowing along the flow path 60 fills the compartments 40 sequentially.

In this embodiment, the compartments 40 are essentially rectangular, preferably with curved corners, and are located side by side. Compared to straight corners, curved corners may reduce the retention of cells when the compartments are emptied. The passageways 90 are provided alternatively on the top side and the bottom side of the container.

The inlet port 10 and the outlet port 20 may be fitted with a Luer lock or another valve fitting. The outlet port 20 may be alternatively or additionally fitted with filters or a sealed compartment under negative pressure. Each compartment 40 may be sized to contain five cubic centimeters of biological fluid. Each compartment 40 may comprise at least one individual port 30, which may be fitted with a Luer lock or other valve. The individual ports 30, which permit access to the compartments 40, may be all located either on the top side of the compartments 40 as shown, or on the bottom side of the compartments 40.

Turning to FIG. 1A, to seal off each container from the others, the passageways 90 may comprise one or more sets 80 of male ribs and female channels located on one side of a separation mark 70, and one or more sets 85 of male ribs and female channels located on the other side of the separation mark 70. The set 80 may provide a first closure along at least one cross section located on one side of the separation mark 70 upon mating of the ribs with the channels. The set 85 may provide a second closure along at least another cross section located on the other side of the separation mark 70 upon mating of the ribs with the channels. The male rib and female channel of each set 80 and 85 may be similar to the ZIPLOC® or GLADLOCK® bag closure.

Alternatively or additionally, the flexible top sheet and flexible bottom sheets may be fused together to seal off each container from the others, for example by applying heat.

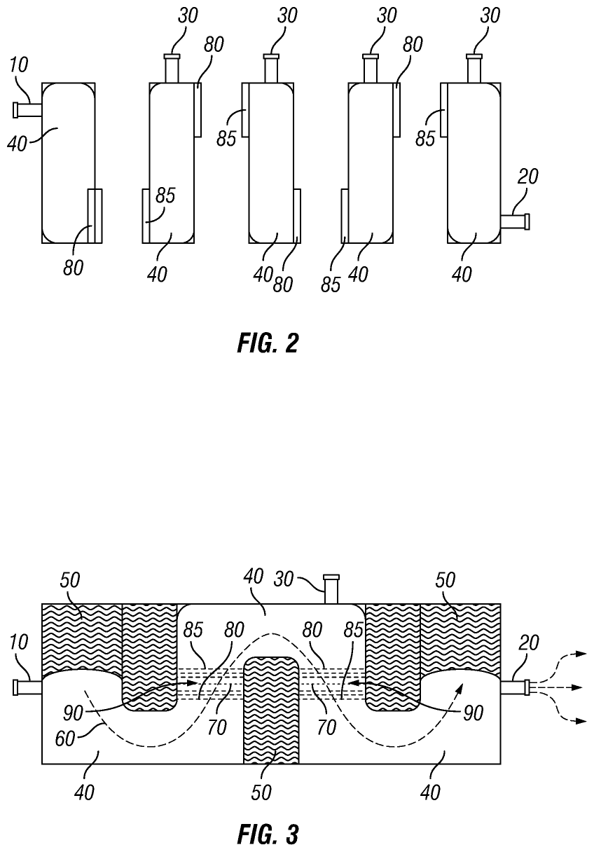

Turning to FIG. 2, the compartments 40 may be separated by severing the container along the separation marks 70. The biological fluid in each compartment 40 thus remains sealed using the Luer lock provided at the individual port 30 or another valve, the closure provided by the set 80, and/or the closure provided by the set 85.

The embodiment of FIG. 3 may be similar to the embodiment of FIG. 2, except that the compartments 40, instead of having an essentially rectangular shape, have a U-shape. Also, the closures between the compartments 40, instead of being vertical, are horizontal.

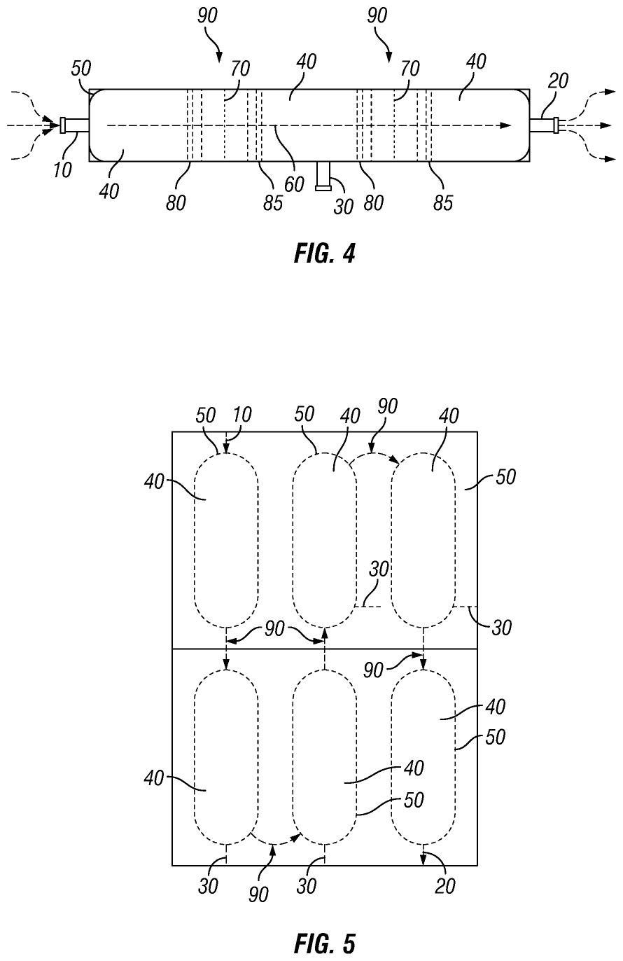

In the embodiment of FIG. 4, all the compartments 40 are aligned, and the flow path 60 is straight.

In the embodiment of FIG. 5, some of the compartments as aligned with the next compartments along the flow path, like in a configuration similar to the configuration shown in FIG. 4, and some compartments 40 are side by side with the next compartments along the flow path, in a configuration similar to the configuration shown in FIG. 1.

In all the configurations shown in FIGS. 1, 3, 4 and 5, the biological fluid flows sequentially into the compartments 40, which are then sealed off after filling, either by pressing, by fusing, or by both pressing and fusing, as explained herein. The compartments 40 may then be separated by severing the storage bag between two adjacent closures, for example along the separation mark 70. The compartments 40 may also be frozen, either before or after separation of the compartments 40.

While the disclosure is susceptible to various modifications and alternative forms, specific embodiments thereof are shown by way of example in the drawings and description. It should be understood, however, that the drawings and detailed description thereto are not intended to limit the claims to the particular form disclosed, but on the contrary, the intention is to cover all modifications, equivalents, and alternatives falling within the scope of the claims.

1. A storage bag for a biological fluid, comprising:
a bottom sheet;
a top sheet superimposed on the bottom sheet; wherein a first portion of the top sheet is sealed against a second, corresponding portion of the bottom sheet, wherein the first portion and second portion enclose a ribbon having a first end and a second end, wherein the ribbon is an essentially flat, narrow and long tubular, wherein the ribbon is continuous between the first end and the second end;
a first port connected across the first end of the ribbon;
a second port connected across the second end of the ribbon;
a first male rib attached to one of the top sheet and the bottom sheet along a first cross section of the ribbon;
a first female channel attached to the other of the top sheet and the bottom sheet along the first cross-section of the ribbon, wherein the first male rib and the first female channel are configured to form a first closure between the top sheet and the bottom sheet upon mating of the first female channel with the first male rib;
a second male rib attached to one of the top sheet and the bottom sheet along a second cross-section of the ribbon, wherein the second cross-section is offset from the first cross-section; and
a second female channel attached to the other of the top sheet and the bottom sheet along the second cross-section of the ribbon, wherein the second male rib and the second female channel are configured to form a second closure upon mating of the second female channel with the second male rib.

2. The storage bag of claim 1, wherein the top sheet and the bottom sheet are flexible.

3. The storage bag of claim 1, wherein the first closure and the second closuare fluid-tight.

4. The storage bag of claim 1, further comprising a filter connected to the second port.

5. The storage bag of claim 1, wherein the first cross-section is adjacent to the second cross-section.

6. The storage bag of claim 1, further comprising a valve connected to the first port or the second port.

7. The storage bag of claim 6, wherein the valve comprises a Luer lock.

8. The storage bag of claim 1, wherein the ribbon forms a single tube.

9. The storage bag of claim 1, wherein the ribbon forms a sequence of elongated compartments, interconnected by passageways.

10. The storage bag of claim 9, wherein any passageway is connected to an extremity of a first elongated compartment and to an extremity of a second elongated compartment.

11. The storage bag of claim 10, wherein any passageway is only connected to the extremity of the first elongated compartment and to the extremity of the second elongated compartment. 12-17. (Canceled)

18. A storage bag for a biological fluid, comprising:
a bottom sheet;
a top sheet superimposed on the bottom sheet; wherein a first portion of the top sheet is sealed against a second, corresponding portion of the bottom sheet, wherein the first portion and second portion enclose a ribbon having a first end and a second end, wherein the ribbon is an essentially flat, narrow and long tubular, wherein the ribbon is continuous between the first end and the second end;
a first port connected across the first end of the ribbon;
a second port connected across the second end of the ribbon;
a first means for closure between the top sheet and the bottom sheet; and
a second means for closure between the top sheet and the bottom sheet, wherein the second means for closure if offset from the first means from closure.

19. The storage bag of claim 18, wherein the ribbon forms a single tube.

20. The storage bag of claim 18, wherein the ribbon forms a sequence of elongated compartments, interconnected by passageways.

21. The storage bag of claim 20, wherein any passageway is connected to an extremity of a first elongated compartment and to an extremity of a second elongated compartment.

22. The storage bag of claim 21, wherein any passageway is only connected to the extremity of the first elongated compartment and to the extremity of the second elongated compartment.

23. A storage container for a biological fluid, comprising:
a sequence of elongated compartments interconnected by passageways, the sequence forming a single continuous tube having a first end and a second end;
a first port connected to the first end of the continuous tube;
a second port connected to the second end of the continuous tube;
a first male rib attached to a wall portion of one of the passageways along a first cross-section of the continuous tube;
a first female channel attached to a complementary wall portion of one of the passageways along the first cross-section of the continuous tube, wherein the first male rib and the first female channel are configured to form a first closure across one of the passageways upon mating of the first female channel with the first male rib;
a second male rib attached to a wall portion of one of the passageways along a second cross-section of the continuous tube, wherein the second cross-section is offset from the first cross-section; and
a second female channel attached to a complementary wall portion of one of the passageways along the second cross-section of the continuous tube, wherein the second male rib and the second female channel are configured to form a second closure across the continuous tube upon mating of the second female channel with the second male rib.

24. (canceled)
